(19) United States
(12) Patent Application Publication
(10) Pub. No.: US 2024/0285403 A1
(43) Pub. Date: Aug. 29, 2024

(54) REPLACEMENT HEART VALVE SYSTEM WITH ANTI-MIGRATION ELEMENT

(71) Applicant: BOSTON SCIENTIFIC SCIMED, INC., Maple Grove, MN (US)

(72) Inventors: Tim O'Connor, Galway (IE); Sean O'Sullivan, Galway (IE); Declan Loughnane, Galway (IE); Joseph Murphy, Galway (IE)

(73) Assignee: BOSTON SCIENTIFIC SCIMED, INC., MAPLE GROVE, MN (US)

(21) Appl. No.: 18/588,457

(22) Filed: Feb. 27, 2024

Related U.S. Application Data
(60) Provisional application No. 63/448,819, filed on Feb. 28, 2023.

(51) Int. Cl. A61F 2/24

(52) U.S. Cl. CPC A61F 2/2436; A61F 2/2412; A61F 2220/0025

(57) ABSTRACT

A replacement heart valve system may include a replacement heart valve implant including an expandable framework configured to shift from a radially collapsed configuration to a radially expanded configuration and a plurality of valve leaflets attached to the expandable framework at a plurality of commissure posts, and an implant delivery system including a handle and an elongate shaft assembly, the elongate shaft assembly including a tubular member fixedly attached to the handle, and an implant holding portion configured to constrain the expandable framework in the radially collapsed configuration. The elongate shaft assembly includes an anti-migration element configured to shift between a delivery configuration and an open configuration. The anti-migration element is configured to prevent proximal movement of the expandable framework relative to the tubular member in the open configuration.

### CROSS-REFERENCE TO RELATED APPLICATIONS

This application claims the benefit of U.S. Provisional Patent Application Ser. No. 63/448,819, filed Feb. 28, 2023, which is incorporated herein by reference.

### TECHNICAL FIELD

The disclosure relates generally to medical devices and more particularly to medical devices that are adapted for implanting stents and medical devices including a stent component.

### BACKGROUND

A wide variety of intracorporeal medical devices have been developed for medical use including, artificial heart valves for repair or replacement of diseased heart valves. The artificial heart valves need to be precisely aligned relative to a native valve annulus when implanted. Of the known medical devices and methods, each has certain advantages and disadvantages. There is an ongoing need to provide alternative medical devices as well as alternative methods for manufacturing and using medical devices.

### SUMMARY

In one example, a replacement heart valve system may comprise a replacement heart valve implant comprising an expandable framework configured to shift from a radially collapsed configuration to a radially expanded configuration, and a plurality of valve leaflets attached to the expandable framework at a plurality of commissure posts; and an implant delivery system comprising a handle and an elongate shaft assembly, the elongate shaft assembly comprising a tubular member fixedly attached to the handle, and an implant holding portion configured to constrain the expandable framework in the radially collapsed configuration. The elongate shaft assembly may further comprise an anti-migration element configured to shift between a delivery configuration and an open configuration. The anti-migration element may be configured to prevent proximal movement of the expandable framework relative to the tubular member in the open configuration. In addition or alternatively to any example disclosed herein, the anti-migration element is engaged with the plurality of commissure posts.

In addition or alternatively to any example disclosed herein, the anti-migration element extends radially outward from the tubular member in the open configuration.

In addition or alternatively to any example disclosed herein, the anti-migration element includes an annular ring and a plurality of legs extending radially outward from the annular ring, the plurality of legs being engaged with the plurality of commissure posts.

In addition or alternatively to any example disclosed herein, the annular ring is fixedly attached to the tubular member.

In addition or alternatively to any example disclosed herein, each leg of the plurality of legs extends radially outward of the plurality of commissure posts.

In addition or alternatively to any example disclosed herein, the annular ring is disposed distal of a proximalmost portion of the plurality of legs when the anti-migration element is engaged with the plurality of commissure posts.

In addition or alternatively to any example disclosed herein, the plurality of legs extends longitudinally alongside the tubular member when the expandable framework is in the radially collapsed configuration.

In addition or alternatively to any example disclosed herein, a replacement heart valve system may comprise a replacement heart valve implant comprising an expandable framework configured to shift from a radially collapsed configuration to a radially expanded configuration, and a plurality of valve leaflets attached to the expandable framework at a plurality of commissure posts; and an implant delivery system comprising a handle and an elongate shaft assembly, the elongate shaft assembly comprising a tubular member fixedly attached to the handle, and an implant holding portion configured to constrain the expandable framework in the radially collapsed configuration. The elongate shaft assembly may further comprise an anti-migration element configured to shift between a delivery configuration and an open configuration. The anti-migration element may be configured to prevent proximal movement of the expandable framework relative to the tubular member when the expandable framework is released from the implant holding portion.

In addition or alternatively to any example disclosed herein, a portion of the anti-migration element abuts the plurality of commissure posts.

In addition or alternatively to any example disclosed herein, the anti-migration element is not attached to the plurality of commissure posts.

In addition or alternatively to any example disclosed herein, the implant delivery system includes a stent holder fixedly attached to the tubular member. The stent holder may be configured to engage a distal portion of the expandable framework when the expandable framework is constrained within the implant holding portion.

In addition or alternatively to any example disclosed herein, the anti-migration element is disposed proximal of the stent holder.

In addition or alternatively to any example disclosed herein, a method of delivering a replacement heart valve implant to a native heart valve may comprise advancing an implant delivery system to a position adjacent the native heart valve, wherein the replacement heart valve implant is constrained within an implant holding portion of the implant delivery system; and deploying the replacement heart valve implant within the native heart valve. The replacement heart valve implant may comprise an expandable framework configured to shift from a radially collapsed configuration to a radially expanded configuration, and a plurality of valve leaflets attached to the expandable framework at a plurality of commissure posts. The implant delivery system may comprise an anti-migration element configured to shift between a delivery configuration and an open configuration. The anti-migration element may be configured to prevent proximal movement of the expandable framework relative to the implant delivery system when the anti-migration element is in the open configuration.

In addition or alternatively to any example disclosed herein, deploying the replacement heart valve implant may further comprise shifting a proximal sheath of the implant delivery system proximally relative to the replacement heart valve implant to release a proximal portion of the expandable framework, thereby permitting the proximal portion of the expandable framework to shift toward the radially expanded configuration and the anti-migration element to shift toward the open configuration; and thereafter, shifting a distal sheath of the implant delivery system distally relative to the replacement heart valve implant to release a distal portion of the expandable framework, thereby permitting the distal portion of the expandable framework to shift toward the radially expanded configuration.

In addition or alternatively to any example disclosed herein, the anti-migration element is fixedly attached to a tubular member of the implant delivery system at an attachment location disposed radially inward of the replacement heart valve implant.

In addition or alternatively to any example disclosed herein, when the replacement heart valve implant is constrained within the implant holding portion, the anti-migration element extends proximally from the attachment location along an outer surface of the tubular member.

In addition or alternatively to any example disclosed herein, the method may further comprise after deploying the replacement heart valve implant within the native heart valve, proximal retraction of the implant delivery system relative to the replacement heart valve implant disengages the anti-migration element from the expandable framework.

In addition or alternatively to any example disclosed herein, after disengaging the anti-migration element from the expandable framework, the anti-migration element is collapsible radially inward to a withdrawal configuration in which the anti-migration element extends distally from the attachment location.

In addition or alternatively to any example disclosed herein, the anti-migration element is biased toward the open configuration.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a replacement heart valve implant;

FIGS. 2-3 illustrate selected aspects of a replacement heart valve system; and

FIGS. 4-5 illustrate selected aspects of an implant delivery system of the replacement heart valve system.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

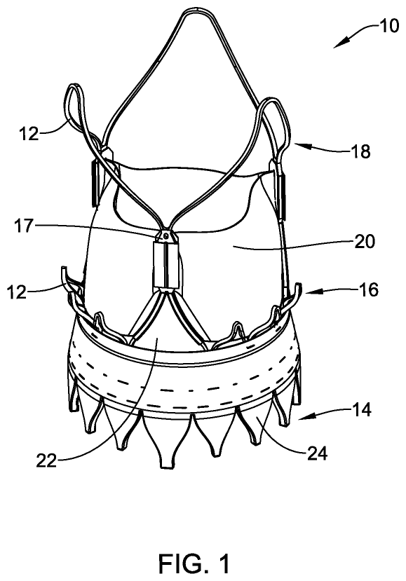

FIG. 1 illustrates selected aspects of a replacement heart valve implant 10. It should be appreciated that the replacement heart valve implant 10 can be any type of replacement heart valve (e.g., a mitral valve, an aortic valve, etc.). In use, the replacement heart valve implant 10 may be implanted (e.g., surgically or through transcatheter delivery) in a mammalian heart. The replacement heart valve implant 10 can be configured to allow one-way flow through the replacement heart valve implant 10 from an inflow end to an outflow end.

For the purpose of this disclosure, the discussion herein is directed toward use in treating a native heart valve such as the aortic valve and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

The replacement heart valve implant 10 may include an expandable framework 12 defining a central lumen. In some embodiments, the expandable framework 12 may have a substantially circular cross-section. In some embodiments, the expandable framework 12 can have a non-circular (e.g., D-shaped, elliptical, etc.) cross-section. Some suitable but non-limiting examples of materials that may be used to form the expandable framework 12, including but not limited to metals and metal alloys, composites, ceramics, polymers, and the like, are described below. The expandable framework 12 may be configured to shift between a radially collapsed configuration and a radially expanded configuration. In some embodiments, the expandable framework 12 may be self-expanding from the radially collapsed configuration to the radially expanded configuration. In some embodiments, the expandable framework 12 may be self-biased toward the radially expanded configuration. In some alternative embodiments, the expandable framework 12 may be mechanically expandable from the radially collapsed configuration to the radially expanded configuration. In some alternative embodiments, the expandable framework 12 may be balloon expandable from the radially collapsed configuration to the radially expanded configuration. Other configurations are also contemplated. In some embodiments, the expandable framework 12 may include and/or define a plurality of interstices (e.g., openings) through the expandable framework 12.

In some embodiments, the expandable framework 12 may include and/or define a lower crown 14 proximate an inflow end, an upper crown 16 proximate an outflow end, and a plurality of stabilization arches 18 extending downstream from the outflow end. In some embodiments, the lower crown 14 may be disposed at the inflow end. In some embodiments, the upper crown 16 may be disposed at the outflow end. In some embodiments, the expandable framework 12 may include a tubular wall defining the central lumen, the inflow end, the outflow end, the lower crown 14, and/or the upper crown 16.

In some embodiments, the expandable framework 12 may include and/or define a plurality of commissure posts 17 proximate the outflow end. In some embodiments, the plurality of commissure posts 17 may at least partially define the outflow end. Other configurations are also contemplated. In some embodiments, the plurality of commissure posts 17 may be disposed longitudinally and/or axially between the upper crown 16 and the plurality of stabilization arches 18. In some embodiments, the plurality of stabilization arches 18 may extend downstream of and/or away from the upper crown 16 and/or the plurality of commissure posts 17 in a direction opposite the lower crown 14. In some embodiments, the upper crown 16 may be disposed longitudinally and/or axially between the lower crown 14 and the plurality of stabilization arches 18. In some embodiments, the upper crown 16 may be disposed longitudinally and/or axially between the lower crown 14 and the plurality of commissure posts 17.

In some embodiments, the replacement heart valve implant 10 may include a proximal portion and a distal portion. In some embodiments, orientation of the replacement heart valve implant 10 may be related to an implant delivery device and/or a direction of implantation relative to a target site. In some embodiments, the proximal portion may include the outflow end and/or the plurality of stabilization arches 18. In some embodiments, the proximal portion may include the plurality of commissure posts 17, the upper crown 16, and/or the plurality of valve leaflets 20. In some embodiments, the distal portion may include the inflow end and/or the lower crown 14. Other configurations are also contemplated.

In some embodiments, the replacement heart valve implant 10 may include a plurality of valve leaflets 20 disposed within the central lumen. The plurality of valve leaflets 20 may be coupled, secured, and/or fixedly attached to the expandable framework 12. In at least some embodiments, the plurality of valve leaflets 20 may be coupled, secured, and/or fixedly attached to the expandable framework 12 at the plurality of commissure posts 17 to form and/or define a plurality of commissures.

Each of the plurality of valve leaflets 20 may include a root edge coupled to the expandable framework 12 and a free edge (e.g., a coaptation edge) movable relative to the root edge to coapt with the free edges of the other valve leaflets along a coaptation region. In some embodiments, the plurality of valve leaflets 20 can be integrally formed with each other, such that the plurality of valve leaflets 20 is formed as a single unitary and/or monolithic unit. In some embodiments, the plurality of valve leaflets 20 may be formed integrally with other structures such as an inner skirt 22 and/or an outer skirt 24, base structures, liners, or the like.

The plurality of valve leaflets 20 may be configured to substantially restrict fluid from flowing through the replacement heart valve implant 10 in a closed position. For example, in some embodiments, the free edges of the plurality of valve leaflets 20 may move into coaptation with one another in the closed position to substantially restrict fluid from flowing through the replacement heart valve implant 10. The free edges of the plurality of valve leaflets 20 may be moved apart from each other in an open position to permit fluid flow through the replacement heart valve implant 10. In FIG. 1, the plurality of valve leaflets 20 is shown in the open position or in a partially open position (e.g., a neutral position) that the plurality of valve leaflets 20 may move to when unbiased by fluid flow.

In some embodiments, the plurality of valve leaflets 20 may be comprised of a polymer, such as a thermoplastic polymer. In some embodiments, the plurality of valve leaflets 20 may include at least 50 percent by weight of a polymer. In some embodiments, the plurality of valve leaflets 20 may be formed from bovine pericardial or other living tissue. Other configurations and/or materials are also contemplated.

In some embodiments, the replacement heart valve implant 10 may include an inner skirt 22 disposed on and/or extending along an inner surface of the expandable framework 12. In at least some embodiments, the inner skirt 22 may be fixedly attached to the expandable framework 12. The inner skirt 22 may direct fluid, such as blood, flowing through the replacement heart valve implant 10 toward the plurality of valve leaflets 20. In at least some embodiments, the inner skirt 22 may be fixedly attached to and/or integrally formed with the plurality of valve leaflets 20. The inner skirt 22 may ensure the fluid flows through the central lumen of the replacement heart valve implant 10 and does not flow around the plurality of valve leaflets 20 when they are in the closed position.

In some embodiments, the replacement heart valve implant 10 can include an outer skirt 24 disposed on and/or extending along an outer surface of the expandable framework 12. In some embodiments, the outer skirt 24 may be disposed at and/or adjacent the lower crown 14. In some embodiments, the outer skirt 24 may be disposed between the expandable framework 12 and the vessel wall in order to prevent fluid, such as blood, flowing around the replacement heart valve implant 10 and/or the expandable framework 12 in a downstream direction. The outer skirt 24 may ensure the fluid flows through the replacement heart valve implant 10 and does not flow around the replacement heart valve implant 10, so as to ensure that the plurality of valve leaflets 20 can stop the flow of fluid when in the closed position.

In some embodiments, the inner skirt 22 may include a polymer, such as a thermoplastic polymer. In some embodiments, the inner skirt 22 may include at least 50 percent by weight of a polymer. In some embodiments, the outer skirt 24 may include a polymer, such as a thermoplastic polymer. In some embodiments, the outer skirt 24 may include at least 50 percent by weight of a polymer. In some embodiments, one or more of the plurality of valve leaflets 20, the inner skirt 22, and/or the outer skirt 24 may be formed of the same polymer or polymers. In some embodiments, the polymer may be a polyurethane. In some embodiments, the inner skirt 22 and/or the outer skirt 24 may be substantially impervious to fluid. In some embodiments, the inner skirt 22 and/or the outer skirt 24 may be formed from a thin tissue (e.g., bovine pericardial, etc.). In some embodiments, the inner skirt 22 and/or the outer skirt 24 may be formed from a coated fabric material. In some embodiments, the inner skirt 22 and/or the outer skirt 24 may be formed from a nonporous and/or impermeable fabric material. Other configurations are also contemplated. Some suitable but non-limiting examples of materials that may be used to form the inner skirt 22 and/or the outer skirt 24 including but not limited to polymers, composites, and the like, are described below. In some embodiments, the inner skirt 22 and/or the outer skirt 24 may seal one of, some of, a plurality of, or each of the plurality of interstices formed in the expandable framework 12. In at least some embodiments, sealing the interstices may be considered to prevent fluid from flowing through the interstices of the expandable framework 12. In some embodiments, the inner skirt 22 and/or the outer skirt 24 may be attached to the expandable framework 12 and/or the plurality of frame struts using one or more methods including but not limited to tying with sutures or filaments, adhesive bonding, melt bonding, embedding or over molding, welding, etc.

In some embodiments, the replacement heart valve implant 10 may include a sealing member disposed on the expandable framework 12 proximate the inflow end. In some embodiments, the sealing member may include and/or may be the inner skirt 22. In some embodiments, the sealing member may include and/or may be the outer skirt 24. In some embodiments, the sealing member may include and/or may be the inner skirt 22 and the outer skirt 24. Other configurations are also contemplated.

In some embodiments, the expandable framework 12 and/or the replacement heart valve implant 10 may have an outer extent of about 23 millimeters (mm), about 25 mm, about 27 mm, about 30 mm, etc. in an unconstrained configuration (e.g., in the radially expanded configuration). In some embodiments, the expandable framework 12 and/or the replacement heart valve implant 10 may have an outer extent of about 10 mm, about 9 mm about 8 mm, about 7 mm, about 6 mm, etc. in the radially collapsed configuration. Other configurations are also contemplated.

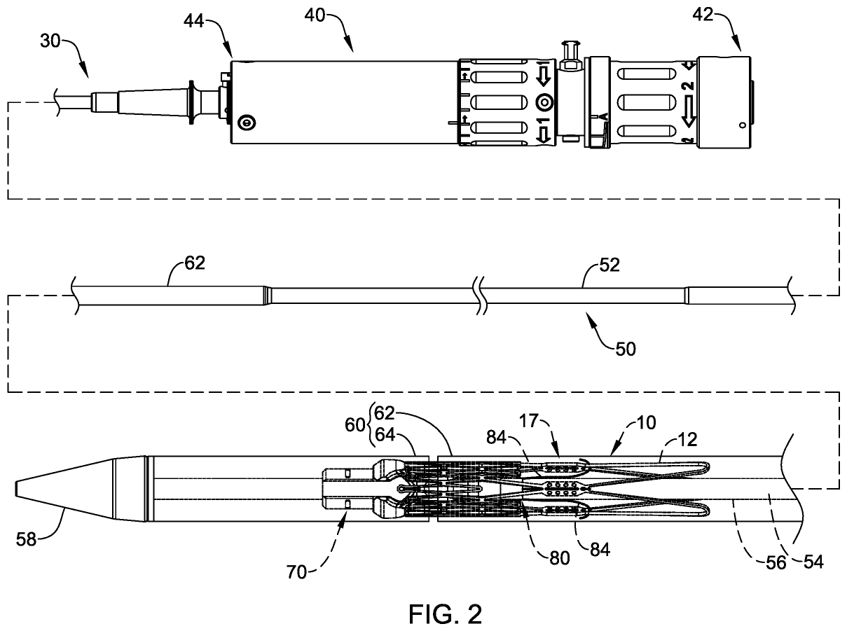

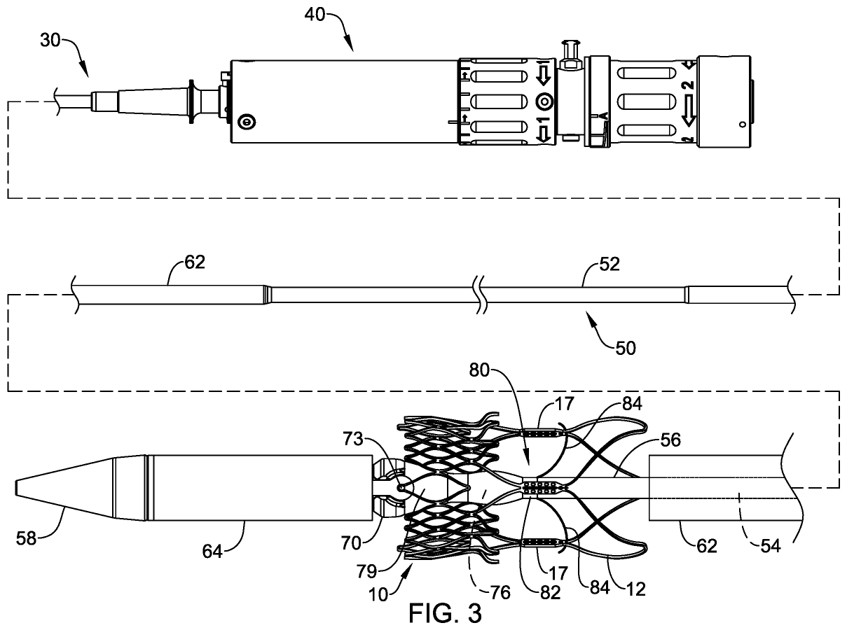

FIG. 2-5 illustrate selected aspects of a replacement heart valve system including the replacement heart valve implant 10 and an implant delivery system 30 for delivering a replacement heart valve implant to a native heart valve (e.g., the aortic valve). The implant delivery system 30 may be compatible with and/or usable with the replacement heart valve implant 10. In FIG. 2, some elements that would be hidden from view are shown in phantom to show relative positioning. In FIGS. 2-3, only the expandable framework 12 of the replacement heart valve implant 10 is shown. Other elements of the replacement heart valve implant 10 have been omitted to improve clarity. It should also be noted that FIGS. 2-3 include at least one change of scale (e.g., all parts of the figure are not drawn to the same scale) to improve viewability and show additional detail of selected aspects of the implant delivery system 30.

The implant delivery system 30 may include a handle 40 and an elongate shaft assembly 50 extending distally from the handle 40. The handle 40 may include a first end 42 and a second end 44 opposite the first end 42. The elongate shaft assembly 50 may extend distally from the second end 44 of the handle 40. The handle 40 may include one or more rotatable knobs. In some embodiments, the one or more rotatable knobs may include a first rotatable knob and a second rotatable knob. In at least some embodiments, the first rotatable knob and/or the second rotatable knob may be configured to rotate about a central longitudinal axis of the implant delivery system 30 and/or the handle 40.

In some embodiments, a distal portion of the implant delivery system 30 and/or the elongate shaft assembly 50 may include an implant holding portion 60 configured to engage with and/or constrain the replacement heart valve implant 10 and/or the expandable framework 12 in the radially collapsed configuration, as seen in FIG. 2. The elongate shaft assembly 50 may comprise a tubular member fixedly attached to the handle 40.

In some embodiments, the elongate shaft assembly 50 may include an outer tubular member 52 extending distally from the handle 40 and an inner shaft 54 extending distally from the handle 40 within the outer tubular member 52 to a distal tip 58 disposed distal of the implant holding portion 60. In some embodiments, the implant holding portion 60 may comprise a proximal sheath 62 and a distal sheath 64. In some embodiments, the proximal sheath 62 and/or the distal sheath 64 may be formed from a polymeric material. In some embodiments, the proximal sheath 62 and/or the distal sheath 64 may include a reinforcing structure disposed therein and/or thereon. In some embodiments, the reinforcing structure may be a coil, a mesh, one or more filaments, bands, or strips, or another suitable structure. Other configurations are also contemplated.

In some embodiments, the inner shaft 54 may be slidably disposed within a lumen of the outer tubular member 52. In some embodiments, the elongate shaft assembly 50 and/or the tubular member fixedly attached to the handle 40 may include an intermediate tubular member 56 disposed within and/or radially inward of the outer tubular member 52 and about and/or radially outward of the inner shaft 54. In some embodiments, the inner shaft 54 may be slidably disposed within a lumen of the tubular member, the outer tubular member 52, and/or the intermediate tubular member 56. In at least some embodiments, the inner shaft 54 and the outer tubular member 52 are each axially translatable relative to the intermediate tubular member 56 independently of each other. For example, the inner shaft 54 may be translated relative to the intermediate tubular member 56 without translating the outer tubular member 52 relative to the intermediate tubular member 56, and vice versa.

In some embodiments, the proximal sheath 62 may be fixedly attached to the outer tubular member 52. In some embodiments, the proximal sheath 62 may be fixedly attached to and/or may extend distally from a distal end of the outer tubular member 52. In some embodiments, the distal sheath 64 and/or the distal tip 58 may be fixedly attached to the inner shaft 54. In some embodiments, the distal sheath 64 may be fixedly attached to the distal tip 58. In some embodiments, the distal sheath 64 may extend proximally from the distal tip 58. In some embodiments, the inner shaft 54 may include and/or at least partially define a guidewire lumen extending therethrough. In some embodiments, the guidewire lumen may extend through the handle 40.

In some embodiments, the handle 40 may be configured to manipulate and/or translate the proximal sheath 62 and/or the distal sheath 64 relative to each other using the first rotatable knob and/or the second rotatable knob. In some embodiments, the handle 40 may be configured to manipulate and/or translate the inner shaft 54 and/or the distal sheath 64 relative to the elongate shaft assembly 50, the outer tubular member 52, the intermediate tubular member 56, and/or the proximal sheath 62. In some embodiments, the handle 40 may be configured to manipulate and/or translate the outer tubular member 52 and/or the proximal sheath 62 relative to the elongate shaft assembly 50, the inner shaft 54, the intermediate tubular member 56, and/or the distal sheath 64. During delivery of the replacement heart valve implant 10 to a treatment site (e.g., the native heart valve, the aortic valve, etc.), the replacement heart valve implant 10 and/or the expandable framework 12 may be disposed at least partially within the proximal sheath 62 and/or the distal sheath 64 in the radially collapsed configuration in a closed configuration of the implant holding portion 60 (e.g., FIG. 2). In some embodiments, the proximal sheath 62 and/or the distal sheath 64 may collectively define the implant holding portion 60 of the implant delivery system 30. In some embodiments, the implant holding portion 60 may be configured to constrain the replacement heart valve implant 10 and/or the expandable framework 12 in the radially collapsed configuration when the implant holding portion 60 is in the closed configuration (e.g., FIG. 2). In some embodiments, the replacement heart valve implant 10 and/or the expandable framework 12 may be releasably coupled to the inner shaft 54, the intermediate tubular member 56, and/or a stent holder 70 (described in more detail below) when the replacement heart valve implant 10 and/or the expandable framework 12 is constrained within the implant holding portion 60 of the implant delivery system 30 in the radially collapsed configuration.

In some embodiments, the proximal sheath 62 may be configured to cover the proximal portion and/or the outflow end of the replacement heart valve implant 10 and/or the expandable framework 12 in the radially collapsed configuration when the implant holding portion 60 is in the closed configuration, and the distal sheath 64 may be configured to cover the distal portion and/or the inflow end of the replacement heart valve implant 10 and/or the expandable framework 12 in the radially collapsed configuration when the implant holding portion 60 is in the closed configuration. In some embodiments, the proximal sheath 62 may be disposed adjacent to the distal sheath 64 in the closed configuration. In some embodiments, the proximal sheath 62 may abut the distal sheath 64 in the closed configuration. In some embodiments, the proximal sheath 62 may be axially spaced apart from the distal sheath 64 in the closed configuration. In some embodiments, the proximal sheath 62 may be axially spaced apart from the distal sheath 64 in the closed configuration by less than 20% of an overall length of the replacement heart valve implant 10 and/or the expandable framework 12. In some embodiments, the proximal sheath 62 may be axially spaced apart from the distal sheath 64 in the closed configuration by less than 15% of an overall length of the replacement heart valve implant 10 and/or the expandable framework 12. In some embodiments, the proximal sheath 62 may be axially spaced apart from the distal sheath 64 in the closed configuration by less than 10% of an overall length of the replacement heart valve implant 10 and/or the expandable framework 12. In some embodiments, the proximal sheath 62 may be axially spaced apart from the distal sheath 64 in the closed configuration by less than 5% of an overall length of the replacement heart valve implant 10 and/or the expandable framework 12. Other configurations are also contemplated.

After advancing the replacement heart valve system and/or the implant delivery system 30 to a position adjacent the native heart valve (e.g., the aortic valve), the replacement heart valve implant 10 and/or the expandable framework 12 may be deployed within the native heart valve (e.g., the aortic valve). Deploying the replacement heart valve implant 10 and/or the expandable framework 12 may include shifting the proximal sheath 62 and the distal sheath 64 of the implant holding portion 60 from the closed configuration to an open configuration, as seen in FIG. 3. In some embodiments, shifting the proximal sheath 62 and the distal sheath 64 of the implant holding portion 60 from the closed configuration to the open configuration may include shifting the proximal sheath 62 and the distal sheath 64 of the implant holding portion 60 axially apart from each other.

In some embodiments, the implant holding portion 60 and/or the elongate shaft assembly 50 may include the stent holder 70, seen in FIG. 3. In at least some embodiments, the stent holder 70 may be fixedly attached to the elongate shaft assembly 50. In some embodiments, the stent holder 70 may be fixedly attached to the intermediate tubular member 56 of the elongate shaft assembly 50. In some embodiments, the stent holder 70 may be integrally formed with the elongate shaft assembly 50 and/or the intermediate tubular member 56. In some embodiments, the stent holder 70 may be configured to engage the expandable framework 12 in the radially collapsed configuration and/or when the replacement heart valve implant 10 is constrained within the implant holding portion 60 of the implant delivery system 30. In some embodiments, the stent holder 70 may include at least one projection 73 configured to engage the expandable framework 12 in the radially collapsed configuration. In some embodiments, the at least one projection 73 may be configured to engage the inflow end of the expandable framework 12 in the radially collapsed configuration. In some embodiments, the at least one projection 73 may extend into and/or through interstices of the expandable framework 12. In some embodiments, the expandable framework 12 may include at least one mounting loop configured to receive and/or engage with the at least one projection 73. Other configurations are also contemplated.

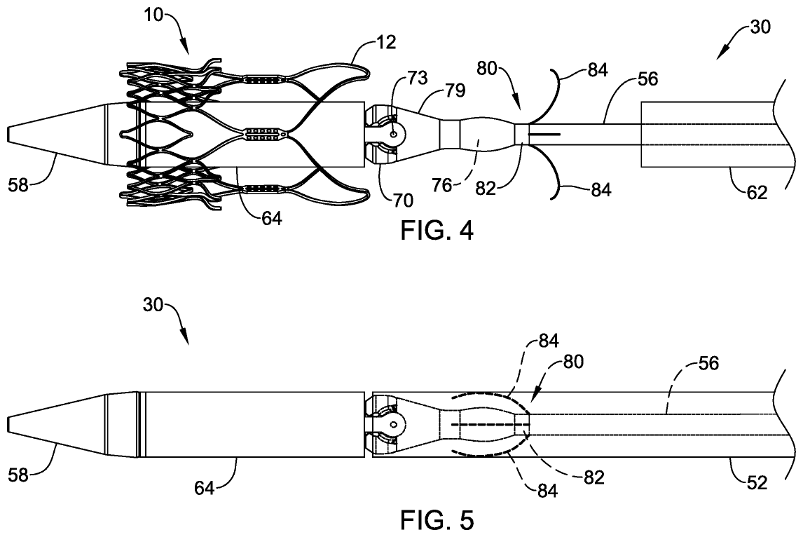

The implant delivery system 30 and/or the elongate shaft assembly 50 may include a primary visual indicator 76 (e.g., FIGS. 3-4) disposed within the replacement heart valve implant 10 when the replacement heart valve implant 10 and/or the expandable framework 12 is constrained within the implant holding portion 60 in the radially collapsed configuration. The primary visual indicator 76 may be configured and/or adapted to be visible under fluoroscopy with an imaging device. Other imaging means suitable for use with transcatheter surgical procedures are also contemplated. The implant delivery system 30 and/or the primary visual indicator 76 may be configured to cooperate with the imaging device to position the replacement heart valve implant 10 at a desired insertion depth within the native heart valve (e.g., the aortic valve). In some embodiments, the primary visual indicator 76 may be fixedly attached to the elongate shaft assembly 50 and/or the intermediate tubular member 56 by a shrink wrap or by an adhesive element. In some embodiments, the primary visual indicator 76 may be and/or may include a marker band. In some embodiments, the primary visual indicator 76 may be at least partially radiopaque. In some embodiments, the primary visual indicator 76 may be completely radiopaque. Other configurations are also contemplated.

In use, the implant delivery system 30 may be advanced to a position adjacent to the treatment site (e.g., the native heart valve). In one example, the implant delivery system 30 may be advanced through the vasculature and across the aortic arch to a position adjacent to the native heart valve (e.g., the aortic valve). Alternative approaches to treat a defective aortic valve and/or other heart valve(s) are also contemplated with the implant delivery system 30.

The desired insertion depth may be selected to maximize radially outward force of the expandable framework 12 within the native heart valve (e.g., the aortic valve). Positioning the replacement heart valve implant 10 at the desired insertion depth and/or within a maximum tolerance from the desired insertion depth, the replacement heart valve implant 10 and/or the expandable framework 12 may exhibit optimal arching within the native heart valve (e.g., the aortic valve) and thereby prevent migration of the replacement heart valve implant 10 and/or the expandable framework 12 downstream (or upstream).

Positioning the replacement heart valve implant 10 and/or the expandable framework 12 within the native heart valve (e.g., the aortic valve) may be accomplished by locating the primary visual indicator 76 relative to the native heart valve (e.g., the aortic valve). During visualization, the native heart valve (e.g., the aortic valve) may be identified and/or visualized under fluoroscopy using known means and/or methods, such as contrast injection.

In some embodiments, the implant delivery system 30 and/or the elongate shaft assembly 50 may include the stent holder 70 configured to engage the expandable framework 12 of the replacement heart valve implant 10 in the radially collapsed configuration and/or when the replacement heart valve implant 10 is constrained within the implant holding portion 60 of the implant delivery system 30. In some embodiments, the stent holder 70 may include a body, a first end portion extending proximally from the body, and a second end portion disposed opposite the first end portion. In some embodiments, at least a portion of the first end portion may extend radially outward from and/or radially outward of the body. In some embodiments, the first end portion may have a generally bulbous shape. In some embodiments, the stent holder 70 may be configured and/or adapted to be visible under fluoroscopy. In some embodiments, the stent holder 70 may be formed from stainless steel. Some suitable but non-limiting materials for the stent holder 70 and/or components or elements thereof are described below.

In some embodiments, an outermost radial extent of the first end portion of the stent holder 70 may be disposed proximate a distal end of the first end portion of the stent holder 70. In some embodiments, the first end portion of the stent holder 70 may be tapered radially inward in a proximal direction from the outermost radial extent of the stent holder 70. In some embodiments, the stent holder 70 may include a lumen extending longitudinally and/or axially therethrough. In at least some embodiments, at least a portion of the elongate shaft assembly 50 may extend longitudinally and/or axially through the lumen of the stent holder 70.

The first end portion may be configured and/or adapted to engage the expandable framework 12 of the replacement heart valve implant 10 in the radially collapsed configuration and/or when the replacement heart valve implant 10 is constrained within the implant holding portion 60 of the implant delivery system 30. In some embodiments, the first end portion may include the at least one projection 73 configured and/or adapted to engage the expandable framework 12 of the replacement heart valve implant 10 in the radially collapsed configuration and/or when the replacement heart valve implant 10 is constrained within the implant holding portion 60 of the implant delivery system 30. In some embodiments, the at least one projection 73 may extend radially outward from the first end portion of the stent holder 70.

In some embodiments, the implant delivery system 30 and/or the implant holding portion 60 may include an atraumatic transition shield 79, seen in FIG. 3. The atraumatic transition shield 79 may be disposed adjacent the stent holder 70. In some embodiments, the atraumatic transition shield 79 may be disposed between the stent holder 70 and the handle 40. In some embodiments, the atraumatic transition shield 79 may be disposed proximal of the stent holder 70. In some embodiments, the atraumatic transition shield 79 may be disposed at and/or adjacent the first end portion of the stent holder 70. In some embodiments, the atraumatic transition shield 79 may axially overlap the first end portion of the stent holder 70. In some embodiments, the atraumatic transition shield 79 may be disposed radially outward of at least a portion of the first end portion of the stent holder 70. In some embodiments, the atraumatic transition shield 79 may be tapered radially inward in the downstream direction and/or the proximal direction and/or toward the handle 40. The atraumatic transition shield 79 may be configured to prevent the replacement heart valve implant 10, the expandable framework 12, the plurality of valve leaflets 20, etc. from catching on the stent holder 70 as the implant delivery system 30 is withdrawn after deploying the replacement heart valve implant 10.

In some embodiments, the primary visual indicator 76 may be disposed adjacent a proximal end of the atraumatic transition shield 79. In some embodiments, the primary visual indicator 76 may be disposed downstream and/or proximal of the atraumatic transition shield 79. In some embodiments, the primary visual indicator 76 and the atraumatic transition shield 79 may axially overlap. In some embodiments, the primary visual indicator 76 may be fixedly attached to the elongate shaft assembly 50. In some embodiments, the primary visual indicator 76 may be embedded in the elongate shaft assembly 50 and/or the intermediate tubular member 56. In some embodiments, the primary visual indicator 76 may be secured and/or fixedly attached to the intermediate tubular member 56, for example by adhesive bonding, welding, shrink wrap, etc. Other configurations are also contemplated.

In some embodiments, the elongate shaft assembly 50 may comprise an anti-migration element 80 configured to shift between a delivery configuration (e.g., FIG. 2) and an open configuration (e.g., FIGS. 3-4). In some embodiments, the anti-migration element 80 may be further configured to shift between the delivery configuration (e.g., FIG. 2), the open configuration (e.g., FIGS. 3-4), and a withdrawal configuration (e.g., FIG. 5).

The anti-migration element 80 may be configured to prevent downstream and/or proximal movement of the replacement heart valve implant 10 and/or the expandable framework 12 relative to the tubular member (e.g., the intermediate tubular member 56) in the open configuration. In at least some embodiments, the anti-migration element 80 may be configured to prevent downstream and/or proximal movement of the replacement heart valve implant 10 and/or the expandable framework 12 relative to the tubular member (e.g., the intermediate tubular member 56) when the replacement heart valve implant 10 and/or the expandable framework 12 is released from the implant holding portion 60.

As seen in FIGS. 2-3, the anti-migration element 80 may be configured to engage with the plurality of commissure posts 17. In some embodiments, the anti-migration element 80 may extend radially outward from the tubular member (e.g., the intermediate tubular member 56). In some embodiments, the anti-migration element 80 may extend radially outward from the tubular member (e.g., the intermediate tubular member 56) in the delivery configuration, the open configuration, and/or the withdrawal configuration. The anti-migration element 80 may be fixedly secured to the tubular member (e.g., the intermediate tubular member 56) at an attachment location.

As seen most clearly in FIG. 4, the anti-migration element 80 may include an annular ring 82 and a plurality of legs 84 extending radially outward from the annular ring 82. As shown in FIGS. 2-3, the plurality of legs 84 of the anti-migration element 80 may be configured to engage with the plurality of commissure posts 17. For example, the plurality of legs 84 of the anti-migration element 80 may be configured to engage with the plurality of commissure posts 17 in the delivery configuration and/or the open configuration. The annular ring 82 of the anti-migration element 80 may be fixedly secured and/or fixedly attached to the tubular member (e.g., the intermediate tubular member 56) at the attachment location. The attachment location may be disposed radially inward of the replacement heart valve implant 10 and/or the expandable framework 12 in the delivery configuration (e.g., when the replacement heart valve implant 10 and/or the expandable framework 12 is constrained within the implant holding portion 60) and/or the open configuration (e.g., as the replacement heart valve implant 10 and/or the expandable framework 12 is being released from the implant holding portion 60). As such, the anti-migration element 80 and/or the annular ring 82 of the anti-migration element 80 is prevented from translating, sliding, etc. along and/or relative to the tubular member (e.g., the intermediate tubular member 56).

Each leg of the plurality of legs 84 of the anti-migration element 80 may extend radially outward from the annular ring 82 of the anti-migration element 80. In at least some embodiments, each leg of the plurality of legs 84 of the anti-migration element 80 may be configured to extend radially outward of the plurality of commissure posts 17. In some embodiments, each leg of the plurality of legs 84 of the anti-migration element 80 may extend radially outward of the plurality of commissure posts 17 in the delivery configuration. In some embodiments, each leg of the plurality of legs 84 of the anti-migration element 80 may extend radially outward of the plurality of commissure posts 17 in the open configuration. In some embodiments, each leg of the plurality of legs 84 of the anti-migration element 80 may extend radially outward of the plurality of commissure posts 17 in the delivery configuration and in the open configuration.

In at least some embodiments, the anti-migration element 80 may be disposed downstream and/or proximal of the stent holder 70 in the delivery configuration and/or the open configuration. In some embodiments, the anti-migration element 80 may be disposed downstream and/or proximal of the atraumatic transition shield 79 in the delivery configuration and/or the open configuration. In some embodiments, the anti-migration element 80 may be disposed downstream and/or proximal of the primary visual indicator 76 in the delivery configuration and/or the open configuration. In some embodiments, the annular ring 82 of the anti-migration element 80 may be disposed downstream and/or proximal of the stent holder 70.

In some embodiments, the annular ring 82 of the anti-migration element 80 may be disposed upstream and/or distal of the plurality of commissure posts 17 in the delivery configuration and/or when the replacement heart valve implant 10 and/or the expandable framework 12 is constrained within the implant holding portion 60. In some embodiments, the annular ring 82 of the anti-migration element 80 may be disposed upstream and/or distal of the plurality of commissure posts 17 in the open configuration (e.g., during release of the replacement heart valve implant 10 and/or the expandable framework 12 from the implant holding portion 60). In some embodiments, the annular ring 82 of the anti-migration element 80 may be disposed upstream and/or distal of a downstreammost end and/or a proximalmost end of the plurality of commissure posts 17 in the delivery configuration and/or when the replacement heart valve implant 10 and/or the expandable framework 12 is constrained within the implant holding portion 60. In some embodiments, the annular ring 82 of the anti-migration element 80 may be disposed upstream and/or distal of the downstreammost end and/or the proximalmost end of the plurality of commissure posts 17 in the open configuration (e.g., during release of the replacement heart valve implant 10 and/or the expandable framework 12 from the implant holding portion 60). As such, in some embodiments, the plurality of commissure posts 17 extends downstream and/or proximal of the annular ring 82 of the anti-migration element 80 in the delivery configuration and/or the open configuration.

In some embodiments, the plurality of legs 84 of the anti-migration element 80 extends downstream and/or proximally from the annular ring 82 of the anti-migration element 80 in the delivery configuration (e.g., FIG. 2) and/or the open configuration (e.g., FIGS. 3-4). In some embodiments, each leg of the plurality of legs 84 of the anti-migration element 80 is coupled, secured, and/or fixedly attached to the annular ring 82 at an attachment end. Each leg of the plurality of legs 84 of the anti-migration element 80 may include a free end opposite the attachment end. In some embodiments, the plurality of legs 84 of the anti-migration element 80 extend downstream and/or proximally from the attachment end in the delivery configuration and/or the open configuration. In some embodiments, the free end may be disposed downstream of and/or proximally of the attachment end and/or the annular ring 82 of the anti-migration element 80 in the delivery configuration. In some embodiments, the free end may be disposed downstream of and/or proximally of the attachment end and/or the annular ring 82 of the anti-migration element 80 in the open configuration. Other configurations are also contemplated.

In some embodiments, a portion of the anti-migration element 80 may abut the plurality of commissure posts 17 in the delivery configuration and/or the open configuration. In some embodiments, the plurality of legs 84 of the anti-migration element 80 may abut the plurality of commissure posts 17 in the delivery configuration and/or the open configuration. In some embodiments, the anti-migration element 80 and/or the plurality of legs 84 of the anti-migration element 80 is in direct contact with the plurality of commissure posts 17 in the delivery configuration and/or the open configuration.

In at least some embodiments, the anti-migration element 80 is not attached to the plurality of commissure posts 17 in the delivery configuration and/or the open configuration. In some embodiments, the anti-migration element 80 is not locked and/or interlocked with the plurality of commissure posts 17 in the delivery configuration and/or the open configuration. In some embodiments, the plurality of legs 84 of the anti-migration element 80 is not attached to the plurality of commissure posts 17 in the delivery configuration and/or the open configuration. In some embodiments, the plurality of legs 84 of the anti-migration element 80 is not locked and/or interlocked with the plurality of commissure posts 17 in the delivery configuration and/or the open configuration.

In some embodiments, the annular ring 82 of the anti-migration element 80 is disposed upstream of a downstreammost portion and/or a downstreammost extent, and/or distal of a proximalmost portion and/or a proximalmost extent, of the plurality of legs 84 of the anti-migration element 80 when the anti-migration element 80 is engaged with the plurality of commissure posts 17. In some embodiments, the annular ring 82 of the anti-migration element 80 is disposed upstream of the downstreammost portion and/or the downstreammost extent, and/or distal of the proximalmost portion and/or the proximalmost extent, of the plurality of legs 84 of the anti-migration element 80 when the anti-migration element 80 is engaged with the plurality of commissure posts 17 in the delivery configuration. In some embodiments, the annular ring 82 of the anti-migration element 80 is disposed upstream of the downstreammost portion and/or the downstreammost extent, and/or distal of the proximalmost portion and/or the proximalmost extent, of the plurality of legs 84 of the anti-migration element 80 when the anti-migration element 80 is engaged with the plurality of commissure posts 17 in the open configuration.

In some embodiments, the plurality of legs 84 of the anti-migration element 80 may extend longitudinally alongside and/or along an outer surface of the tubular member (e.g., the intermediate tubular member 56) when the replacement heart valve implant 10 and/or the expandable framework 12 is constrained in the radially collapsed configuration. In some embodiments, the plurality of legs 84 of the anti-migration element 80 may extend downstream and/or proximally alongside and/or along the outer surface of the tubular member (e.g., the intermediate tubular member 56) when the replacement heart valve implant 10 and/or the expandable framework 12 is constrained in the radially collapsed configuration. In some embodiments, the plurality of legs 84 of the anti-migration element 80 may extend longitudinally alongside and/or along the outer surface of the tubular member (e.g., the intermediate tubular member 56) when the anti-migration element 80 is in the delivery configuration, as seen in FIG. 2. In some embodiments, the plurality of legs 84 of the anti-migration element 80 may extend downstream and/or proximally alongside and/or along the outer surface of the tubular member (e.g., the intermediate tubular member 56) when the anti-migration element 80 is in the delivery configuration.

In some embodiments, the anti-migration element 80 and/or the plurality of legs 84 of the anti-migration element 80 may be biased toward the open configuration. In at least some embodiments, the anti-migration element 80 and/or the plurality of legs 84 of the anti-migration element 80 may be self-biased toward the open configuration. As such, absent external forces on the anti-migration element 80 and/or the plurality of legs 84 of the anti-migration element 80, the anti-migration element 80 and/or the plurality of legs 84 of the anti-migration element 80 will “settle” into the open configuration. In some embodiments, the anti-migration element 80 and/or the plurality of legs 84 of the anti-migration element 80 may be formed from a shape memory material that is heat set in the open configuration. Other configurations are also contemplated.

In some embodiments, the anti-migration element 80 and/or the plurality of legs 84 of the anti-migration element 80 may be biased toward the open configuration by one or more springs. In some embodiments, the anti-migration element 80 and/or the plurality of legs 84 of the anti-migration element 80 may be biased toward the open configuration using mechanical means. Other configurations are also contemplated.

FIG. 5 illustrates selected aspects of the implant delivery system 30 wherein the anti-migration element 80 is disposed in the withdrawal configuration. After release of the replacement heart valve implant 10 and/or the expandable framework 12 from the implant holding portion 60, the implant holding portion 60 may be shifted toward and/or to the closed configuration prior to removing the implant delivery system 30 from the patient. As such, after release of the replacement heart valve implant 10 and/or the expandable framework 12 from the implant holding portion 60, the proximal sheath 62 and the distal sheath 64 may be moved axially and/or longitudinally towards each other. After disengaging the anti-migration element 80 and/or the plurality of legs 84 of the anti-migration element 80 from the replacement heart valve implant 10, the expandable framework 12, and/or the plurality of commissure posts 17, the anti-migration element 80 and/or the plurality of legs 84 of the anti-migration element 80 may be collapsible radially inward to the withdrawal configuration. As the proximal sheath 62 is moved upstream and/or distally toward the distal sheath 64, the proximal sheath 62 may engage the plurality of legs 84 of the anti-migration element 80 and shift the plurality of legs 84 radially inward and in an upstream direction and/or a distal direction such that at least a portion of the anti-migration element 80 extends upstream and/or distally from the attachment location.

In some embodiments, the plurality of legs 84 of the anti-migration element 80 extends upstream and/or distally from the annular ring 82 of the anti-migration element 80 in the withdrawal configuration (e.g., FIG. 5). In some embodiments, the plurality of legs 84 of the anti-migration element 80 extend upstream and/or distally from the attachment end in the withdrawal configuration. In some embodiments, the free end may be disposed upstream of and/or distally of the attachment end and/or the annular ring 82 of the anti-migration element 80 in the withdrawal configuration. Other configurations are also contemplated.

In some embodiments, the plurality of legs 84 of the anti-migration element 80 may extend longitudinally alongside and/or along the outer surface of the tubular member (e.g., the intermediate tubular member 56) when the anti-migration element 80 is in the withdrawal configuration, as seen in FIG. 5. In some embodiments, the plurality of legs 84 of the anti-migration element 80 may extend upstream and/or distally alongside and/or along the outer surface of the tubular member (e.g., the intermediate tubular member 56) when the anti-migration element 80 is in the withdrawal configuration.

A method of delivering the replacement heart valve implant 10 to a native heart valve (e.g., the aortic valve) may comprise advancing the implant delivery system 30 to a position adjacent the native heart valve (e.g., the aortic valve). As discussed herein, the replacement heart valve implant 10 may be constrained within the implant holding portion 60 of the implant delivery system 30, as seen in FIG. 2. The method of delivering the replacement heart valve implant 10 to the native heart valve (e.g., the aortic valve) may further comprise deploying the replacement heart valve implant 10 and/or the expandable framework 12 within the native heart valve (e.g., the aortic valve).

As discussed herein, the implant delivery system 30 may comprise the anti-migration element 80 configured to shift between the delivery configuration and the open configuration. In some embodiments, the anti-migration element 80 may be further configured to shift to the withdrawal configuration. The anti-migration element 80 may be configured to prevent downstream and/or proximal movement of the replacement heart valve implant 10 and/or the expandable framework 12 relative to the implant delivery system 30 and/or the tubular member (e.g., the intermediate tubular member 56) when the anti-migration element 80 is in the open configuration.

In some embodiments, deploying the replacement heart valve implant 10 and/or the expandable framework 12 within the native heart valve (e.g., the aortic valve) may further comprise shifting the proximal sheath 62 of the implant delivery system 30 and/or the implant holding portion 60 downstream and/or proximally relative to the replacement heart valve implant 10 and/or the expandable framework 12 to release the proximal portion of the replacement heart valve implant 10 and/or the expandable framework 12, thereby permitting the proximal portion of the replacement heart valve implant 10 and/or the expandable framework 12 to shift toward the radially expanded configuration and the anti-migration element 80 to shift toward the open configuration. In some embodiments, deploying the replacement heart valve implant 10 and/or the expandable framework 12 within the native heart valve (e.g., the aortic valve) may further comprise thereafter, shifting the distal sheath 64 of the implant delivery system 30 and/or the implant holding portion 60 upstream and/or distally relative to the replacement heart valve implant 10 and/or the expandable framework 12 to release the distal portion of the replacement heart valve implant 10 and/or the expandable framework 12, thereby permitting the distal portion of the replacement heart valve implant 10 and/or the expandable framework 12 to shift toward the radially expanded configuration, as seen in FIG. 3.

In some embodiments, after deploying the replacement heart valve implant 10 and/or the expandable framework 12 within the native heart valve (e.g., the aortic valve), the method may comprise retraction and/or withdrawal of the implant delivery system 30 relative to the replacement heart valve implant 10 and/or the expandable framework 12. In some embodiments, retraction and/or withdrawal of the implant delivery system 30 may include moving and/or translating the implant delivery system 30 downstream and/or proximally relative to the replacement heart valve implant 10 and/or the expandable framework 12. In some alternative configurations, retraction and/or withdrawal of the implant delivery system 30 may include moving and/or translating the implant delivery system 30 upstream and/or distally relative to the replacement heart valve implant 10 and/or the expandable framework 12. Other configurations are also contemplated.

In some embodiments, retraction and/or withdrawal of the implant delivery system 30 relative to the replacement heart valve implant 10 and/or the expandable framework 12 may disengage the anti-migration element 80 and/or the plurality of legs 84 of the anti-migration element 80 from the replacement heart valve implant 10, the expandable framework 12, and/or the plurality of commissure posts 17, as seen in FIG. 4. In some embodiments, the implant delivery system 30 may be retracted and/or withdrawn relative to the replacement heart valve implant 10 and/or the expandable framework 12 by at least a length of the plurality of legs 84 of the anti-migration element 80 as measured from the attachment end to the free end. In some embodiments, the implant delivery system 30 may be retracted and/or withdrawn relative to the replacement heart valve implant 10 and/or the expandable framework 12 by less than the length of the plurality of legs 84 of the anti-migration element 80 as measured from the attachment end to the free end. For example, the implant delivery system 30 may be retracted and/or withdrawn relative to the replacement heart valve implant 10 and/or the expandable framework 12 far enough to permit the anti-migration element 80 and/or the plurality of legs 84 of the anti-migration element 80 to be shifted radially inward toward the withdrawal configuration, wherein the free end of each leg of the plurality of legs 84 of the anti-migration element 80 does not contact the replacement heart valve implant 10, the expandable framework 12, and/or the plurality of commissure posts 17 as the anti-migration element 80 and/or the plurality of legs 84 of the anti-migration element 80 is shifted radially inward toward the withdrawal configuration. Other configurations are also contemplated. In FIG. 4, the implant delivery system 30 is shown retracted and/or withdrawn relative to the replacement heart valve implant 10 and/or the expandable framework 12 by an exaggerated amount in order to show selected features of the implant delivery system 30 more clearly.

In some embodiments, after deploying the replacement heart valve implant 10 and/or the expandable framework 12 within the native heart valve (e.g., the aortic valve), the method may comprise shifting the implant holding portion 60 from the open configuration to the closed configuration. For example, shifting the implant holding portion 60 from the open configuration to the closed configuration may include moving and/or translating the proximal sheath 62 and the distal sheath 64 towards each other. In some embodiments, shifting the implant holding portion 60 from the open configuration to the closed configuration may include moving and/or translating the proximal sheath 62 upstream and/or distally relative to the tubular member (e.g., the intermediate tubular member 56) and/or moving and/or translating the distal sheath 64 downstream and/or proximally relative to the tubular member (e.g., the intermediate tubular member 56).

In some embodiments, after disengaging the anti-migration element 80 and/or the plurality of legs 84 of the anti-migration element 80 from the replacement heart valve implant 10, the expandable framework 12, and/or the plurality of commissure posts 17, the anti-migration element 80 and/or the plurality of legs 84 of the anti-migration element 80 is collapsible radially inward to the withdrawal configuration in which the anti-migration element 80 and/or the plurality of legs 84 of the anti-migration element 80 extends upstream and/or distally from the annular ring 82 of the anti-migration element 80 and/or the attachment location of the annular ring 82 of the anti-migration element 80 to the tubular member (e.g., the intermediate tubular member 56).

In some embodiments, shifting the implant holding portion 60 from the open configuration to the closed configuration may include shifting the anti-migration element 80 and/or the plurality of legs 84 of the anti-migration element 80 toward and/or to the withdrawal configuration, as seen in FIG. 5. In some embodiments, shifting the implant holding portion 60 from the open configuration to the closed configuration may include shifting and/or collapsing the anti-migration element 80 and/or the plurality of legs 84 of the anti-migration element 80 radially inward toward and/or to the withdrawal configuration.

In some embodiments, after deploying the replacement heart valve implant 10 and/or the expandable framework 12 within the native heart valve (e.g., the aortic valve), the method may comprise further retraction and/or withdrawal of the implant delivery system 30 relative to the replacement heart valve implant 10 and/or the expandable framework 12. In some embodiments, after deploying the replacement heart valve implant 10 and/or the expandable framework 12 within the native heart valve (e.g., the aortic valve), the method may comprise retraction and/or withdrawal of the implant delivery system 30 from the treatment site, from the position adjacent the native heart valve (e.g., the aortic valve), and/or from the patient.

In at least some interventions, the replacement heart valve implant 10 may be deployed within the native heart valve (e.g., the native heart valve is left in place and not excised). Alternatively, the native heart valve may be removed (such as through valvuloplasty, for example) and the replacement heart valve implant 10 may be deployed in its place as a replacement.

The materials that can be used for the various components of the replacement heart valve system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the replacement heart valve implant, the expandable framework, the plurality of valve leaflets, the implant delivery system, the handle, the elongate shaft assembly, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly (alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine);

antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A replacement heart valve system comprising:
a replacement heart valve implant comprising:
an expandable framework configured to shift from a radially collapsed configuration to a radially expanded configuration; and
a plurality of valve leaflets attached to the expandable framework at a plurality of commissure posts; and
an implant delivery system comprising a handle and an elongate shaft assembly, the elongate shaft assembly comprising a tubular member fixedly attached to the handle, and an implant holding portion configured to constrain the expandable framework in the radially collapsed configuration;
wherein the elongate shaft assembly further comprises an anti-migration element configured to shift between a delivery configuration and an open configuration;
wherein the anti-migration element is configured to prevent proximal movement of the expandable framework relative to the tubular member in the open configuration.

2. The replacement heart valve system of claim 1, wherein the anti-migration element is engaged with the plurality of commissure posts.

3. The replacement heart valve system of claim 1, wherein the anti-migration element extends radially outward from the tubular member in the open configuration.

4. The replacement heart valve system of claim 3, wherein the anti-migration element includes an annular ring and a plurality of legs extending radially outward from the annular ring, the plurality of legs being engaged with the plurality of commissure posts.

5. The replacement heart valve system of claim 4, wherein the annular ring is fixedly attached to the tubular member.

6. The replacement heart valve system of claim 4, wherein each leg of the plurality of legs extends radially outward of the plurality of commissure posts.

7. The replacement heart valve system of claim 4, wherein the annular ring is disposed distal of a proximalmost portion of the plurality of legs when the anti-migration element is engaged with the plurality of commissure posts.

8. The replacement heart valve system of claim 4, wherein the plurality of legs extends longitudinally alongside the tubular member when the expandable framework is in the radially collapsed configuration.

9. A replacement heart valve system comprising:
a replacement heart valve implant comprising:
an expandable framework configured to shift from a radially collapsed configuration to a radially expanded configuration; and
a plurality of valve leaflets attached to the expandable framework at a plurality of commissure posts; and
an implant delivery system comprising a handle and an elongate shaft assembly, the elongate shaft assembly comprising a tubular member fixedly attached to the handle, and an implant holding portion configured to constrain the expandable framework in the radially collapsed configuration;
wherein the elongate shaft assembly further comprises an anti-migration element configured to shift between a delivery configuration and an open configuration;
wherein the anti-migration element is configured to prevent proximal movement of the expandable framework relative to the tubular member when the expandable framework is released from the implant holding portion.

10. The replacement heart valve system of claim 9, wherein a portion of the anti-migration element abuts the plurality of commissure posts.

11. The replacement heart valve system of claim 9, wherein the anti-migration element is not attached to the plurality of commissure posts.

12. The replacement heart valve system of claim 9, wherein the implant delivery system includes a stent holder fixedly attached to the tubular member, the stent holder being configured to engage a distal portion of the expandable framework when the expandable framework is constrained within the implant holding portion.

13. The replacement heart valve system of claim 12, wherein the anti-migration element is disposed proximal of the stent holder.

14. A method of delivering a replacement heart valve implant to a native heart valve, comprising:
advancing an implant delivery system to a position adjacent the native heart valve,
wherein the replacement heart valve implant is constrained within an implant holding portion of the implant delivery system; and
deploying the replacement heart valve implant within the native heart valve;
wherein the replacement heart valve implant comprises an expandable framework configured to shift from a radially collapsed configuration to a radially expanded configuration, and a plurality of valve leaflets attached to the expandable framework at a plurality of commissure posts;
wherein the implant delivery system comprises an anti-migration element configured to shift between a delivery configuration and an open configuration;
wherein the anti-migration element is configured to prevent proximal movement of the expandable framework relative to the implant delivery system when the anti-migration element is in the open configuration.

15. The method of claim 14, wherein deploying the replacement heart valve implant further comprises:
shifting a proximal sheath of the implant delivery system proximally relative to the replacement heart valve implant to release a proximal portion of the expandable framework, thereby permitting the proximal portion of the expandable framework to shift toward the radially expanded configuration and the anti-migration element to shift toward the open configuration; and
thereafter, shifting a distal sheath of the implant delivery system distally relative to the replacement heart valve implant to release a distal portion of the expandable framework, thereby permitting the distal portion of the expandable framework to shift toward the radially expanded configuration.

16. The method of claim 14, wherein the anti-migration element is fixedly attached to a tubular member of the implant delivery system at an attachment location disposed radially inward of the replacement heart valve implant.

17. The method of claim 16, wherein when the replacement heart valve implant is constrained within the implant holding portion, the anti-migration element extends proximally from the attachment location along an outer surface of the tubular member.

18. The method of claim 17, further comprising:
after deploying the replacement heart valve implant within the native heart valve, proximal retraction of the implant delivery system relative to the replacement heart valve implant disengages the anti-migration element from the expandable framework.

19. The method of claim 18, wherein after disengaging the anti-migration element from the expandable framework, the anti-migration element is collapsible radially inward to a withdrawal configuration in which the anti-migration element extends distally from the attachment location.

20. The method of claim 14, wherein the anti-migration element is biased toward the open configuration.
